(19) United States
(12) Patent Application Publication
(10) Pub. No.: US 2018/0318573 A1
(43) Pub. Date: Nov. 8, 2018

(54) ROTATABLE TUBING CONNECTOR FOR BREASTMILK SYSTEMS

(71) Applicant: MEDELA HOLDING AG, Baar (CH)

(72) Inventor: Joseph Wach, Ingleside, IL (US)

(21) Appl. No.: 15/967,458

(22) Filed: Apr. 30, 2018

Related U.S. Application Data
(60) Provisional application No. 62/501,485, filed on May 4, 2017.

(51) Int. Cl. A61M 39/10; A61M 1/06; F16L 11/04; F16L 27/02

(52) U.S. Cl. CPC A61M 39/1055; F16L 27/023; F16L 11/04; A61M 1/062

(57) ABSTRACT

A rotatable connector for a breastmilk system. The connector can be customized to provide a range of rotatable motion based on a selected size, shape and material. The connector can be designed to allow a wide range of motion, as needed, during use. The connector can be located near or at least partially within a connecting component of the system, as desired. Some, or all, of the material of the connector can be designed to rotate in response to forces applied to the connector during use.

### CROSS-REFERENCE TO RELATED APPLICATIONS

This application claims priority to U.S. Provisional Patent Application No. 62/501,485, filed on May 4, 2017, entitled “Rotatable Tubing Connector for Breastmilk Systems,” the entire disclosure of which is hereby incorporated by reference.

### FIELD OF THE DISCLOSURE

The present disclosure relates generally to a connector configured to provide a rotatable connection between components of a breastmilk system.

### BACKGROUND

Known connectors that connect components of breast milk systems, such as breastpumps, can include tight, friction fit connectors that are designed to reduce leakage of pressure generating fluid from the system, such as from the interface between tubing elements and connection points to a pressure generating component of the system. However, these fit connections can cause stress on connecting components. Connecting components can eventually fail and break due to stress incurred during usage based on forces applied to tubing, for example.

### SUMMARY

A rotatable connector for a breastmilk system is set forth. The connector can be customized to provide a range of rotatable motion based on a selected size, shape and material. The connector can be designed to allow a wide range of motion, as needed, during use. The connector can be located near or at least partially within a connecting component of the system, as desired. Some or all material of the connector can be designed to be rotatable, ie to move, pivot, or rotate in response to forces applied to the connector during use. At least one section of the rotatable connector can be configured to form a rotatable connection. Alternatively, the entire connector can be configured to form a rotatable connection.

Various advantages of the present disclosure are specifically described below in reference to the exemplary embodiments, or conceptually embodied therein. The drawings and description herein are provided to merely illustrate examples of the general concepts discussed throughout the present disclosure. Numerous changes and modifications can be made, as known to those of skill in the art, without departing from the general principles set forth herein. In addition, all patents and publications referenced are incorporated herein by reference in the entirety.

### BRIEF DESCRIPTION OF THE DRAWINGS

These and other features and advantages of the various exemplary embodiments disclosed herein will be better understood with respect to the following description and drawings, in which:

FIG. 1 is an exemplary embodiment of a rotatable connector for a breastmilk system showing both pivoting and rotatable capabilities to reduce fatigue on the breastmilk system components;

FIG. 2 is an exemplary embodiment of another rotatable connector for a breastmilk system where the connector is partially disposed in a connecting component of the breastmilk system;

FIG. 3 is an exemplary embodiment of another rotatable connector for a breastmilk system, where the connector is disposed near the connecting component of the breastmilk system;

FIG. 4 is an exemplary embodiment of yet another rotatable connector for a breastmilk system formed of a continuous material;

FIG. 5 is an exemplary embodiment of yet another rotatable connector for a breastmilk system formed of a discontinuous material.

FIG. 6 is an exemplary embodiment of yet another rotatable connector for a breastmilk system wherein a rotatable section of the connector is selectively connectable to a tubing component of the system.

FIG. 7 illustrates an exemplary embodiment of yet another rotatable connector for a breastmilk system wherein a female connector end is configured for insertion into the tubing, and the tubing can move or rotate relative to a male connector end of the system selectively seated in the female end of the rotatable connector.

Common reference numerals are used throughout the drawings and the detailed description to indicate the same elements.

### DETAILED DESCRIPTION

The detailed description set forth below in connection with the appended drawings is intended as a description of certain exemplary embodiments of various system components constructed in accordance with the principles herein, including. These examples are not intended to represent the only embodiments or forms that may be developed or utilized according to these principles.

Certain aspects of some embodiments constructed in accordance with the principles herein are directed toward a connector having a movable, pivotal and/or rotatable portion that absorbs forces on the system during use. The rotatable portion can provide a force reducing movement and/or a movable cushion between an interface component and a connecting component, such as tubing, for example, during operation of a breastmilk system. The rotatable portion can reduce stress from forces applied to the interface component during operation of the breastmilk system.

As will be described in more detail below, several embodiments are contemplated in accordance with the principles herein, some of which are shown in the exemplary embodiments.

It is understood that the breastmilk systems described herein may be used to deliver a wide range of fluid contents via fluid transmission components connectable to the rotatable connector, such as air, water, breastmilk, medicines, or other fluids. Such contents will be collectively referred to herein as “fluid” for purposes of simplicity.

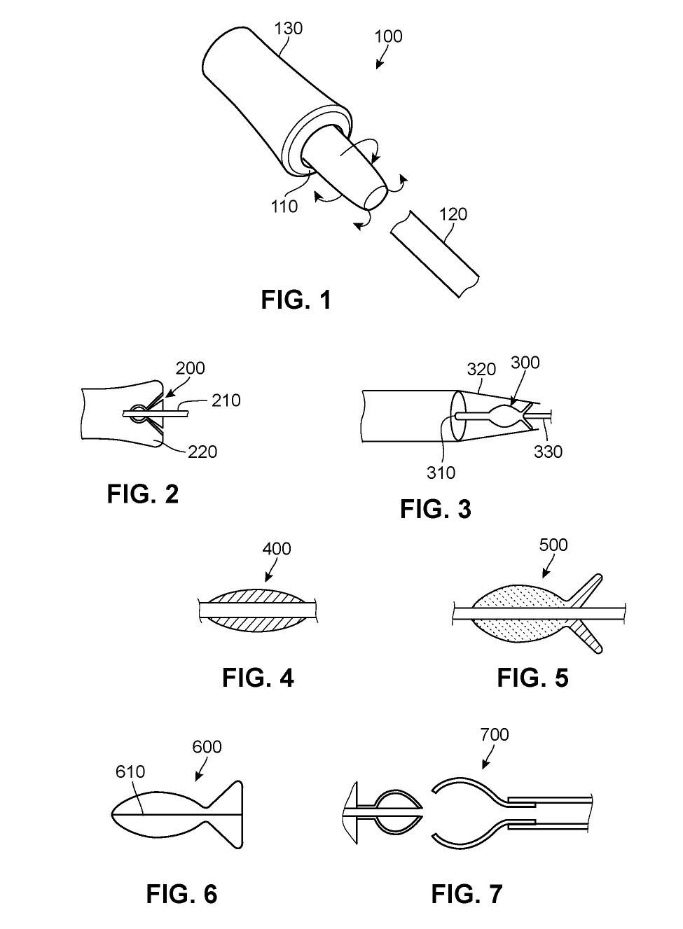

An exemplary embodiment of a rotatable connector constructed in accordance with the principles herein is shown generally at 100 in FIG. 1. The rotatable connector 100 can take a suitable shape that facilitates various movements with respect to a tubing 120 selectively connected to the system, such as a spherical shape relative to a connection port, or any other suitable shape. The rotatable connector 100 can be configured such that at least one rotatable section 110 of the rotatable connector 100 can move, rotate or pivot when forces are applied to an associated tubing 120 into which the rotatable connection is seated (not shown). In the embodiment of FIG. 1, the at least one rotatable section 110 is configured to move relative to a section 130 of the connector 100 when connected to tubing 120 and when forces are then applied to the tubing 120.

Another exemplary embodiment of a rotatable connector constructed in accordance with the principles herein is shown generally at 200 in FIG. 2, wherein the entire rotatable connector 200 is configured to rotate. A tubing 210, or other suitable connecting component can be connected to or seated in the rotatable connector 200. More than one rotatable connector can be provided and connected to tubing. The tubing can then connect various components of the breastmilk system together. For example, rotatable connectors can be provided near or at both ends of the tubing to reduce fatigue at both the pump and the kit connections within the system without compromising the hermetic seal of the system, since vacuum integrity is critical in a breastmilk system. The rotatable connector 200 can be configured to provide a movable connection within an interface component 220, allowing the tubing 210 to apply reduced forces to any connecting components in the interface 220 due to the cushioning effect of the rotatable connecting component 200.

Yet another exemplary embodiment of a rotatable connector constructed in accordance with the principles herein is shown generally at 300 in FIG. 3. Here the rotatable connector 300 is located near a connecting component 310 of a breastmilk system component. The rotatable connector 300 reduces strain otherwise imposed on the interior wall of connection port 320 when the tubing 330 is pulled during operation of the breastmilk system.

Still another exemplary embodiment of a rotatable connector is set forth in FIG. 4. The rotatable connector 400 can be formed of any suitable continuous exterior surface material that facilitates rotation of the connector within the system. The connector 400 can be formed of multiple materials, or of a single material that facilitates rotation within the system.

Yet another exemplary embodiment of a rotatable connector is set forth in FIG. 5, and shown generally at 500. Here small sections of the connector rotate and/or are formed of a movable material that allows for absorption of certain torsion forces that can arise due to pulling on or movement of the tubing during operation of the breastmilk system.

Various additional features can be included in a rotatable connector, such as the example shown generally at 600 in FIG. 6, to facilitate easy application or change of a rotatable connector 600 in a breastmilk system. For example, a slit 610 can be provided along an axis of the rotatable connector and extending to an outer surface to enable opening of the rotatable connector and connection to a connecting component, such as tubing, of the system.

In yet another embodiment illustrated in FIG. 7, the rotatable connector is formed by seating a female end of the connector in tubing, and selectively connecting the tubing to a male connector end of the system. In this embodiment, the tubing is able to pivot, move and/or rotate outside of the connection head, rather than partially within a connection port.

Variations of the specific device configurations shown and described herein that provide a are within the scope of the principles of the present disclosure, and are included in all claims deriving therefrom.

What is claimed is:

1. A connector configured to connect breastmilk system components comprising:
a rotatable connector section selectively connectable to a tubing component of the breastmilk system.

2. The connector of claim 1, the rotatable connector section disposed near a connecting component of the breastmilk system.

3. The connector of claim 1, the rotatable connector section disposed partially within the connecting component of the system.

4. The connector of claim 1, the rotatable connector section formed of a continuous material.

5. The connector of claim 1, the rotatable connector section formed of a discontinuous material.

6. The connector of claim 1, wherein the range of motion of the rotatable connector section is based on at least one of a selected size, shape and material.

7. The connector of claim 1, wherein a preselected section of the connector is designed to move in response to forces applied to the connector during use via a tubing component connected to the connector.

8. A breastmilk system comprising:
a rotatable connector provided on a tubing component, the rotatable connector configured to reduce tension on components of the breastmilk system connected to the tubing.

9. A fatigue-reducing connector for a breastmilk pumping system comprising:
a fatigue-reducing section configured to move in response to an applied tension force; and
a tubing component, directly or indirectly connected to the connector, and configured to be movable and to generate the applied tension force when moved relative to the connector between a first position and a second position during operation of the breastmilk pumping system.

10. The fatigue-reducing connector of claim 9, the connector further including a substantially circular cross section.
